(19) United States
(12) Patent Application Publication
(10) Pub. No.: US 2022/0092339 A1
(43) Pub. Date: Mar. 24, 2022

(54) APPARATUS FOR CLASSIFYING MEDICAL IMAGE

(71) Applicant: Vingroup Joint Stock Company, Ha Noi City (VN)

(72) Inventors: Quan M. Tran, Ha Noi City (VN); Huy D. Ta, Ha Noi City (VN); Thanh M. Huynh, Ha Noi City (VN); Nam H. Nguyen, Ha Noi City (VN); Phuong-Anh T. Nguyen, Ha Noi City (VN); Steven QH. Truong, Ha Noi City (VN)

(21) Appl. No.: 17/393,656

(22) Filed: Aug. 4, 2021

(30) Foreign Application Priority Data
Sep. 23, 2020 (VN) 1-2020-05475

(51) Int. Cl. G06K 9/62; G06T 7/00; G16H 30/20

(52) U.S. Cl. CPC G06K 9/628; G06T 7/0012; G06T 2207/20081; G16H 30/20; G06T 2207/20084; G06K 9/6234

(57) ABSTRACT

Provided is an apparatus for classifying a medical image. The apparatus includes a database configured to store a first image, a generator configured to generate a second image on the basis of a latent vector which is a concatenation of noise information having a certain size and random uniform class labels of a plurality of diseases, a discriminator configured to receive the first image and the second image and attempt to recognize the first image and the second image as a real image and a fake image, and a classifier configured to classify the first image and the second image.

### CROSS-REFERENCE TO RELATED APPLICATION

This application claims priority to Vietnamese Application No. 1-2020-05475 filed on Sep. 23, 2020. The aforementioned application is incorporated herein by reference in its entirety.

### TECHNICAL FIELD

The present invention relates to an apparatus for classifying a medical image on the basis of machine learning.

### RELATED ART

The current worldwide outbreak of the new coronavirus COVID-19 (coronavirus disease 2019; the pathogen called SARS-CoV-2; previously 2019-nCoV) has now spread across 213 countries and territories. Globally, 9.2 million people have been infected with more than 473,000 deaths as of late June 2020.

A standard golden method to diagnose COVID-19 is Reverse Transcription-Polymerase Chain Reaction (RT-PCR). However, due to the sampling collection procedure, this method may not capture the appearance of COVID-19 well. Therefore, from filtering, classification, and detection of COVID-19 to examinations and treatments, all suffer from the contagious properties of viruses and pose considerable challenges while being applied on a massive scale. Studies and reports from around the world show that COVID-19 has a variety of clinical manifestations, ranging from asymptomatic infection or just a common cold to severe illnesses that cause acute respiratory damage, multiple organ failure, and can lead to death if not treated promptly. At present, the RTPCR molecular biology test to look for specific genes of the virus is a valid test to confirm the diagnosis of infection with a sensitivity of 60% to 70% and a specificity of 95% to 100%.

However, there are still 30% to 40% of false negative cases of COVID-19 patients with negative RT-PCR results. Chest X-ray (CXR) and Computed Tomography (CT) play a particularly important role in screening and diagnosis suggestions. Besides, recent studies also show the essential values of CXR and CT in the diagnosis. The specificity of CXR diagnosis is 69%, and the specificity of Chest CT can be up to 98%. Also, Chest CT is not only valuable in the diagnosis of COVID-19 but also significant in monitoring disease progression and evaluating treatment effects.

Medical image-assisted diagnostics, such as X-ray and Computed Tomography (CT), alongside with RT-PCR, become essential to examining the people. Among them, CXR tends to be feasible due to its quick scanning time and sterilization. CXR is one of the most popular diagnostic imaging procedures over the world, estimating roughly two billion scans per year. It comes with ease in installing for local hospitals or even portability with a medical truck. Nevertheless, the image features or indicators of COVID-19 symptoms on CXR can be missed because of a variety of contrasts, scanning angles; or due to the radiologists' reading (mainly noise from years of experience and/or domains of expertise). These drawbacks can be avoided by using deep neural networks that learn statistically from the data and perform consistently as long as there are enough image samples to be trained.

### SUMMARY

The present invention is directed to providing an apparatus for classifying a medical image on the basis of machine learning by which accuracy in disease diagnosis may be improved by generating a large number of medical images of a specific disease from a few medical images.

Objectives to be achieved by embodiments of the present invention are not limited thereto, and the present invention may also include objectives or effects which can be derived from solutions or embodiments described below.

According to an aspect of the present invention, there is provided an apparatus for classifying a medical image, the apparatus including: a database configured to store a first image, a generator configured to generate a second image on the basis of a latent vector which is a concatenation of noise information having a certain size and random uniform class labels of a plurality of diseases, a discriminator configured to receive the first image and the second image and attempt to recognize the first image and the second image as a real image and a fake image, and a classifier configured to classify the first image and the second image.

The noise information may have a size of 16 dimensions and may be generated on the basis of a normal distribution.

The random uniform class labels of the plurality of diseases may be random uniform class labels of coronavirus disease 2019 (COVID-19), airspace opacity, consolidation, and pneumonia and may have a value of 0 for negative cases of the diseases and a value of 1 for positive cases of the diseases.

The discriminator may calculate a probability distribution with relation to the second image.

The generator and the discriminator may be implemented as progressive growing generative adversarial networks (GANs).

The classifier may be implemented as DenseNet121.

In the classifier, the number of output neurons may be set differently depending on a classification type.

The classifier may set the number of the output neurons to 1 when the classification type is a binary label classification and set the number of the output neurons to 4 when the classification type is a multi-label classification.

All of the activations of the classifier and the discriminator are replaced by Leaky ReLU.

The classifier may set a leaky coefficient to 0.02.

A final layer of the classifier may use a logistic sigmoid function.

The generator, the discriminator, and the classifier may be trained on the basis of the following formula:

$${min}_{{θ}_{G} , {θ}_{C}} {max}_{{θ}_{D}} L ( C ) + λ ( V ( G , D ) + L ( G , C ) )$$

where L(C) denotes a classification loss, V(G, D) denotes an adversarial loss, L(G, C) denotes a classification-driven generative loss, and λ denotes a hyperparameter.

The hyperparameter may be 0.1.

The hyperparameter may be 1 when optimizing discriminator and generator.

The apparatus may further include a diagnostic unit configured to make a disease diagnosis from an image of a patient on the basis of the classified first image and second image.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a block diagram of an apparatus for classifying a medical image according to an exemplary embodiment of the present invention;

FIG. 2 is a conceptual diagram of the apparatus for classifying a medical image according to the exemplary embodiment of the present invention;

FIGS. 3A to 3D show simulation results of the apparatus for classifying a medical image according to the exemplary embodiment of the present invention;

FIGS. 4A and 4B show simulation results of an apparatus for classifying a medical image according to another exemplary embodiment of the present invention;

FIG. 5 shows a set of simulation results of an apparatus for classifying a medical image according to still another exemplary embodiment of the present invention; and

FIG. 6 shows a set of simulation results of an apparatus for classifying a medical image according to yet another exemplary embodiment of the present invention.

### DETAILED DESCRIPTION

Although a variety of modifications and several embodiments of the present invention can be made, exemplary embodiments will be shown in the accompanying drawings and described. However, it should be understood that the present invention is not limited to the specific embodiments and includes all changes, equivalents, or substitutions within the spirit and technical scope of the present invention.

The terms including ordinal numbers, such as second and first, may be used for describing a variety of elements, but the elements are not limited by the terms. The terms are used only for distinguishing one element from another element. For example, without departing from the scope of the present invention, a second element may be referred to as a first element, and similarly, a first element may be referred to as a second element. The term “and/or” includes any combination of a plurality of associated listed items or any one of the plurality of associated listed items.

When it is stated that one element is “connected” or “joined” to another element, it should be understood that the element may be directly connected or joined to the other element but still another element may be present therebetween. On the other hand, when it is stated that one element is “directly connected” or “directly joined” to another element, it should be understood that no other element is present therebetween.

Terms used herein are used only for describing the specific embodiments and are not intended to limit the present invention. Singular expressions include plural expressions unless clearly defined otherwise in context. Throughout this specification, it should be understood that the terms “include,” “have,” etc. are used herein to specify the presence of stated features, numbers, steps, operations, elements, parts, or combinations thereof but do not preclude the presence or addition of one or more other features, numbers, steps, operations, elements, parts, or combinations thereof.

Unless defined otherwise, terms used herein including technical or scientific terms have the same meanings as terms which are generally understood by those of ordinary skill in the art. Terms such as those defined in commonly used dictionaries should be construed as having meanings consistent with contextual meanings of related art and should not be interpreted in an idealized or excessively formal sense unless clearly defined so herein.

Hereinafter, embodiments of the present invention will be described in detail with reference to the accompanying drawings. Throughout the drawings, like reference numerals will be given to the same or corresponding elements, and a repeated description thereof will be omitted.

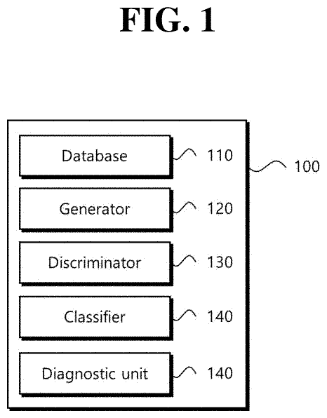

FIG. 1 is a block diagram of an apparatus for classifying a medical image according to an exemplary embodiment of the present invention.

Referring to FIG. 1, an apparatus 100 for classifying a medical image according to the exemplary embodiment of the present invention may include a database 110, a generator 120, a discriminator 130, a classifier 140, and a diagnostic unit 150.

The database 110 may store first images. The first images may be medical images of patients with specific diseases. According to the exemplary embodiment of the present invention, the first images may be X-ray images of the chests of patients infected with pneumonia, consolidation, airspace opacity and coronavirus disease 2019 (COVID-19). The first images may include images captured during a treatment process of the specific diseases. For example, the first images may include images captured in a treatment process for patients infected with pneumonia, consolidation, airspace opacity and COVID-19.

The database 110 may include personal information corresponding to the first image. The personal information may include genders and ages. The database 110 may include pandemic declaration, clinical information (symptoms and temperatures), and reverse-transcription polymerase chain reaction (RT-PCR) tests corresponding to the first images.

The generator 120 may generate second images on the basis of latent vectors.

A latent vector may be a vector which is a concatenation of noise information having a certain size and random uniform class labels of a plurality of diseases.

Noise information may be extracted from a normal distribution. The normal distribution may be generated on the basis of the first images stored in the database 110. The noise information may have the certain size. According to the exemplary embodiment of the present invention, the size of the noise information may be 16.

The random uniform class labels of the plurality of diseases may be those of COVID-19, airspace opacity, consolidation, and pneumonia. In other words, the plurality of diseases may be COVID-19, airspace opacity, consolidation, and pneumonia. The random uniform class labels may have a value of 0 for negative cases of the diseases and a value of 1 for positive cases of the diseases.

According to the exemplary embodiment of the present invention, a latent vector may be a high-dimensional vector obtained by adding noise information and the dimensions of random uniform class labels of a plurality of diseases. For example, when the size of noise information is 16 and the number of the plurality of diseases are four, the latent vector may be a high-dimensional feature vector of 20 dimensions.

The discriminator 130 may receive the first images and the second images and attempt to recognize the first images and the second images as real images and fake images. The discriminator 130 attempts to differentiate the real images that have been drawn from the database 110, i.e., a distribution (Pₓ) and the fake ones produced by the generator 120. The discriminator 130 may calculate a probability distribution with relation to the second images.

The classifier 140 may classify the first images and the second images. In an embodiment, the classifier 140 may classify the first images and the second images on the basis of labels of the first images and the second images. To this end, the classifier 140 may receive the first images from the database 110 and receive the second images from the generator 120. The labels may include first labels and second labels. The first labels may be labels input by a user to correspond to the first images or the second images. In this case, the user may be an expert in the technical field of the first images and the second images. For example, the user may be a doctor. The second labels may be labels previously allocated to the first images or the second images. For example, the second labels of the first images may be labels stored in the database 110, and the second labels of the second images may be labels based on the random uniform class labels.

The classifier 140 does its regular job to contrast the types of disease in images, both from being manually annotated by doctors and from pre-assigned label generated ones. The mechanism behind the apparatus of invention is to enrich the image samples, in which the label can be controlled in a much broader distribution. By treating the handful labeled data as a subset of the above bundle, the noisy labeling from doctors (mainly coming from different years of experiences, domain expertise, etc.) can be suppressed.

The diagnostic unit 150 may make a disease diagnosis from an image of a patient on the basis of the classified first images and second images. For example, the diagnostic unit 150 may diagnose the patient with a disease by comparing a probability distribution with relation to the first images and the second images with the probability distribution of an image of the patient.

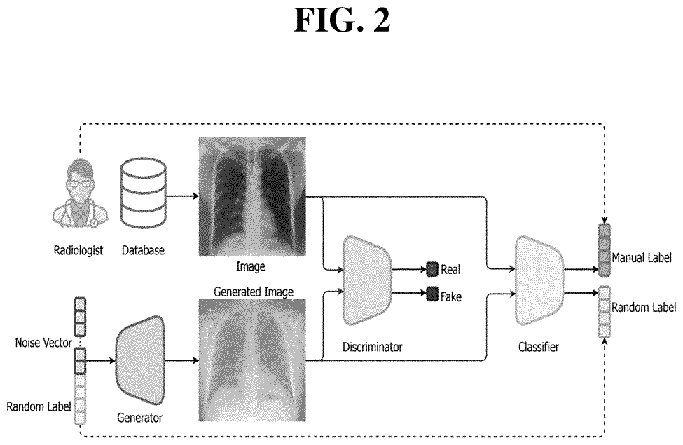

FIG. 2 is a conceptual diagram of the apparatus for classifying a medical image according to the exemplary embodiment of the present invention.

The present invention proposes a novel generative deep-learning-based model to classify COVID-19 chest X-ray images. For example, chest X-ray images may be COVID-19 chest X-ray images. The present invention may also be referred to as a Virtual laBel Generative Adversarial Network (VBGAN).

Referring to FIG. 2, the present invention includes a generation model which generates an image through adversarial training of the generator and the discriminator which are multilayer perceptrons. The generator may be a differentiable function which is a multilayer perceptron having a weight θ_{G} as a parameter. Also, the discriminator may be a function which is a multilayer perceptron outputting a single scalar and having a weight θ_{D} as a parameter. The discriminator may represent a probability that input data is obtained from an actual distribution or latent space. The generator may receive a random noise vector z to generate data. By determining whether the generated data is real or fake, the generator may be trained to deceive the discriminator while generating data similar to real data. The discriminator may be trained to better discriminate.

The apparatus for classifying a medical image according to the exemplary embodiment of the present invention may be described as a process for finding a weight of a minimax problem as in Expression 1 below.

The weight may include a first weight, a second weight, and a third weight.

The first weight θ_{G} may denote a weight corresponding to the generator, the second weight θ_{c} may denote a weight corresponding to the classifier, and the third weight θ_{D} may denote a weight corresponding to the discriminator.

$${min}_{{θ}_{G} , {θ}_{C}} {max}_{{θ}_{D}} L ( C ) + λ ( V ( G , D ) + L ( G , C ) ) \qquad [ Expression 1 ]$$

where L(C) denotes a classification loss, V(G, D) denotes an adversarial loss, and L(G, C) denotes a classification-driven generative loss.

The classification loss may be defined as in Expression 2 below.

$$L ( C ) = {𝔼}_{x \sim {P}_{x}} [ {\sum }_{c} - p ( c \vert x ) log C ( c \vert x ) ] \qquad [ Expression 2 ]$$

When pathology c is included in the image x, p(c|x)=1.

The adversarial loss may be defined as in Expression 3 below.

$$V ( G , D ) = {𝔼}_{\begin{matrix}z \sim 𝒩 \\ c \sim {𝒰}_{{P}_{c}}\end{matrix}} [ log ( 1 - D ( G ( z , c ) ) ] + {𝔼}_{x \sim {P}_{x}} [ log D ( x ) ] \qquad [ Expression 3 ]$$

The classification-driven generative loss L(G, C) may be defined as in Expression 4 below.

$$L ( G , C ) = {𝔼}_{\begin{matrix}z \sim 𝒩 \\ c \sim {𝒰}_{{P}_{c}}\end{matrix}} [ - log C ( c \vert G ( z , c ) ) ] \qquad [ Expression 4 ]$$

The loss function of Expression 1 may be broken down into several terms and used in updating the generator, the discriminator, and the classifier. It is noted that maximizing a third weight θ_{D} may be equivalent to minimizing the same minus amount of energy.

$$\begin{matrix}{ℒ}_{gen} = λ ( L ( G , C ) + V ( G , D ) ) = λ ( {𝔼}_{\begin{matrix}z \sim 𝒩 \\ c \sim {𝒰}_{{P}_{c}}\end{matrix}} [ - log C ( c \vert G ( z , c ) ) ] + {𝔼}_{\begin{matrix}z \sim 𝒩 \\ c \sim {𝒰}_{{P}_{c}}\end{matrix}} [ log ( 1 - D ( G ( z , c ) ) ) ] ) & [ Expression 5 ] \\ {ℒ}_{dis} = - λ V ( G , D ) = - λ ( {𝔼}_{x \sim {P}_{x}} [ log D ( x ) ] + {𝔼}_{\begin{matrix}z \sim 𝒩 \\ c \sim {𝒰}_{{P}_{c}}\end{matrix}} [ log ( 1 - D ( G ( z , c ) ) ) ] ) & [ Expression 6 ]\end{matrix}$$

$${ℒ}_{cls} = L ( C ) + λ L ( G , C ) = {𝔼}_{x \sim {P}_{x}} [ {\sum }_{c} - p ( c \vert x ) log C ( c \vert x ) ] + λ {𝔼}_{\begin{matrix}z \sim 𝒩 \\ c \sim {𝒰}_{{P}_{c}}\end{matrix}} [ - log C ( c \vert G ( z , c ) ) ] \qquad [ Expression 7 ]$$

In Expressions 5 to 7, λ may denote a hyperparameter. The hyperparameter may be previously set by the user. The hyperparameter may have various values. For example, the hyperparameter may have any one value among 0.5, 0.2, 0.1, and 0.01. Preferably, the hyperparameter have a value of 0.1. When the hyperparameter has a value of greater than 0.1, too much noise is caused at the beginning of the training, which may make it difficult for the classifier to converge. On the contrary, when the hyperparameter has a value of less than 0.1, normalization weakens, and the classifier may overfit on training data.

The present invention adopts the Progressive Growing GAN architectures for the generator and discriminator. For the classifier, the present invention chooses DenseNet121, and the present invention sets the appropriate number of output neurons (1 for binary classification, and 4 for multi-label classification). All of the activations of the classifier and the discriminator are replaced by Leaky ReLU.

For the classifier, the present invention sets a leaky coefficient (α) to 0.02 (as opposed to 0.2 in general GAN settings) so that it does not deviate much from the original structure while still allowing the gradient to flow to the generator. For the last layer of the classifier, the logistic sigmoid function is used.

The present invention first trains the generator and the discriminator without label conditioning on the training set to get appropriate second images (chest X-ray (CXR) images). After the generator converges, the present invention attaches the classifier to the scheme and a two layers sub-network, which acts as mapping from label concatenated noise to latent space before inputting it through the generator. The present invention then jointly trains all of these for additional 100 epochs with the cosine learning rate decay for the classifier.

Adam optimizer in “Adam: A method for stochastic optimization,” by D. P. Kingma and J. Ba, is used with a default learning rate of 0.001 for the discriminator, generator, and classifier. For discriminator and generator, the similar training hyper-parameters in “Unsupervised representation learning with deep convolutional generative adversarial networks,” by A. Radford, L. Metz, and S. Chintala, is used and β₁ is set to 0 and β₂ is set to 0.99. Due to the imbalance of COVID positive instances in the training set, these instances are upsampled to the same amount of negative examples.

For hyperparameter λ, the present invention experiments with a variety of values: 0.5, 0.2, 0.1, and 0.01. The present invention discovered that value 0.1 works best because bigger values lead to too much noise at the beginning of the training, which makes the classifier hard to converge. In comparison, lower values lead to a weak regularization, and the classifier overfits on training data. In addition, the losses of the generator (Expression 5) and the discriminator (Expression 6) are directly proportional to λ. Therefore, λ is set to be equal to 1 when optimizing the discriminator and generator for faster convergence.

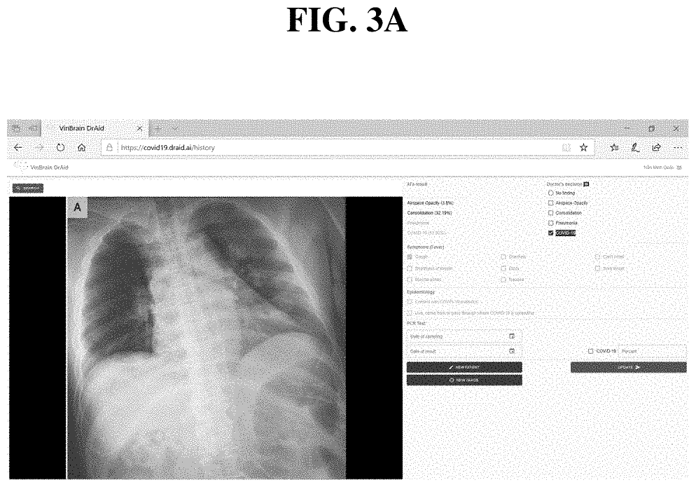

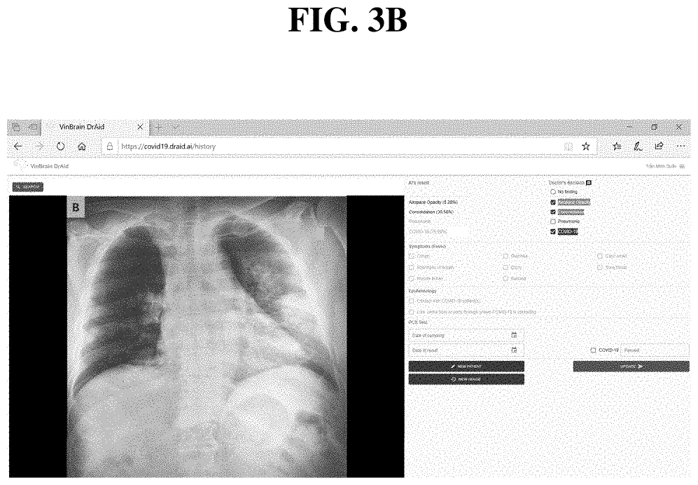

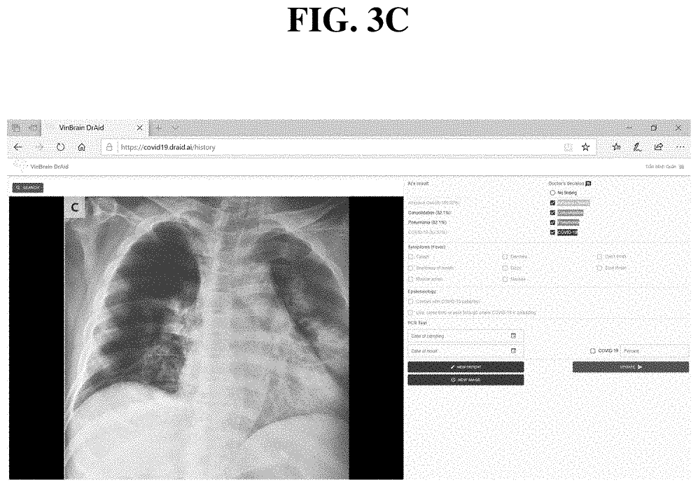

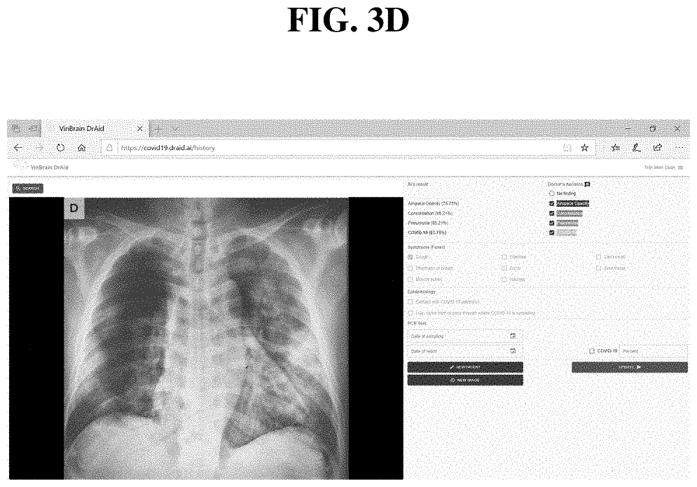

FIGS. 3A to 3D show simulation results of the apparatus for classifying a medical image according to the exemplary embodiment of the present invention.

FIGS. 3A to 3D show classification results obtained by inputting chest X-ray images of COVID-19 patients to the apparatus for classifying a medical image according to the exemplary embodiment of the present invention.

In FIGS. 3A to 3D, the chest X-ray images of the COVID-19 patients are captured during a certain time period after the patients are hospitalized.

As shown in FIGS. 3A to 3D, the probabilities that COVID-19 appears in the images increase from 57.92% (at admission stage A, FIG. 3A) to 76.99% (stage B, FIG. 3B), 82.57% (stage C, FIG. 3C) and 93.75% (stage D, FIG. 3D) a few days after.

This prediction aligns with the severely increasing symptoms of airspace opacity, consolidation, and pneumonia, in addition to other clinical symptoms (fever, cough, shortness of breath, muscle aches) in the medical reports.

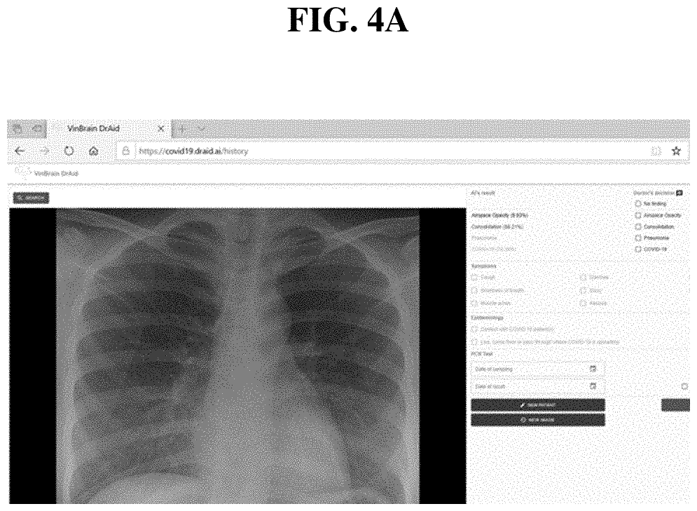

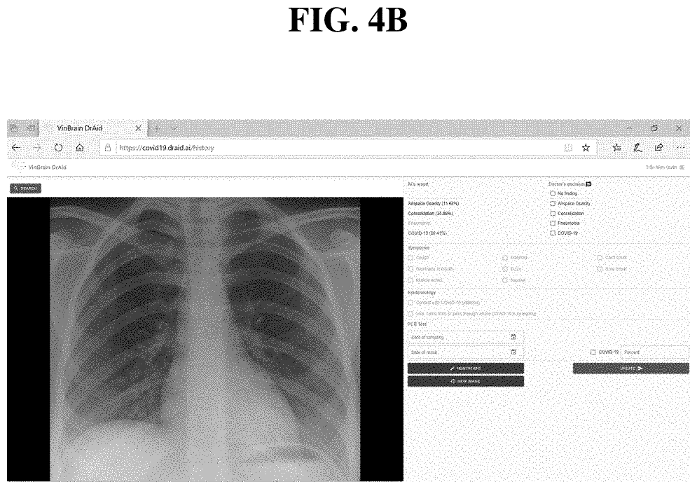

FIGS. 4A and 4B show simulation results of an apparatus for classifying a medical image according to another exemplary embodiment of the present invention.

COVID-19 can cause a wide range of symptoms: people in an early stage of infection can show no symptoms at all but they can already spread the coronavirus. FIGS. 4A and 4B illustrate two images that have been missed by doctors' screenings. Although there are no clinical symptoms such as high temperatures, cough, shortness of breath, and the like, the apparatus of invention can send out the warnings that these patients have potentially infected by COVID-19 (76.39% and 80.41%) and need prompt action. Their RT-PCR results after that also confirm the positive statuses.

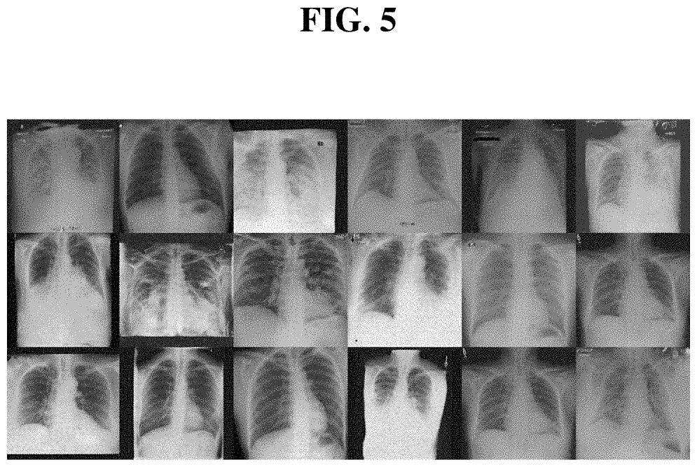

FIG. 5 shows a set of simulation results of an apparatus for classifying a medical image according to still another exemplary embodiment of the present invention.

Chest X-ray images can be generated from the generator by inputting a random positive/negative label and a random normal noise vector. Since the pixel values of images generated by VBGAN generator lie in the range of [−1, 1], the present invention can normalize it by using each image min and max values. FIG. 5 presents some random chest X-ray images of people who do not exist that are synthesized by a bunch of random noise latent vectors. The present invention can plan to construct a gamification labeling tool (a training environment) that shuffles these generated images (their labels are known) and real images to regularize the decisions from doctors. This makes the final readings more sharp and precise.

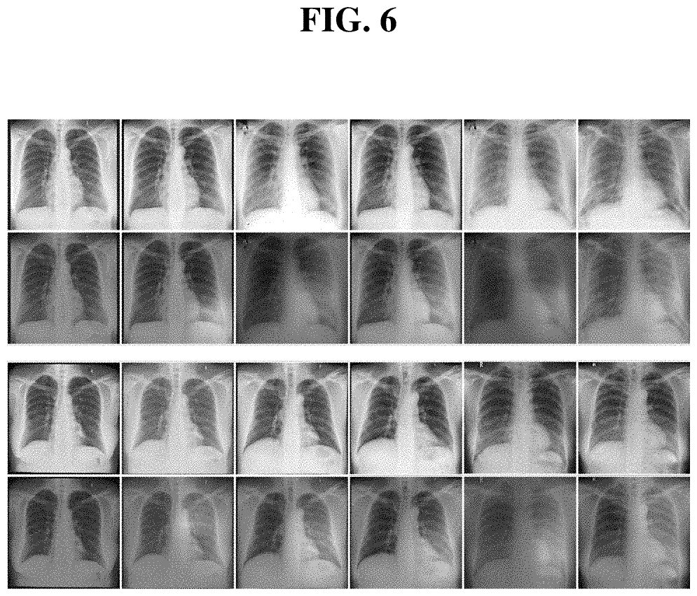

FIG. 6 shows a set of simulation results of an apparatus for classifying a medical image according to yet another exemplary embodiment of the present invention.

The present invention can further investigate how COVID-19 evolves through time by interpolating in the latent space.

The present invention can start from a random noise vector sampled from the standard normal distribution and negative COVID label values (0). Next, the present invention can increase the label value from negative (0) to positive (1) with a step of 0.2 while keeping the noise vector fixed. As can be seen in FIG. 5, when COVID-19 probability increases from negative to positive, the areas around the chest's border become increasingly foggy similar to ground-glass opacity which shows the effects of the novel coronavirus. From left to right, these synthesized images have clear observations of increasing the lung damage, which is indicated by their associated heat maps (produced by a standard GradCAM) and which is confirmed by doctors.

Comparison results between the present invention (VBGAN) and other apparatuses will be described below with reference to Tables 1 and 2.

### A. Evaluation Metrics

The standard evaluation is used for statistical classification in machine learning such as its confusion matrix's derivations: Precision, Recall, Fl Score, etc, beyond the Accuracy to measure the effectiveness of the proposed model in intrasetups for ablation study and comparison with other work. The meaning of the chosen evaluation metrics is summarized in Table 1. Since the data distribution is highly imbalanced, the F1 score becomes an important metric while harmonizing the high Precision (or Positive Predictive Value) and the Sensitivity (or Recall) due to not wanting to miss the positive cases but still wanting to accurately classify them.

TABLE 1

| Metrics | Abbreviation/Formula |
|---|---|
| Conditional Positive | P |
| Conditional Negative | N |
| True Positive | TP |
| True Negative | TN |
| False Positive | FP |
| False Negative | FN |
| True Positive Rate (Sensitivity, | $TPR = \frac{TP}{P} = \frac{TP}{TP + FN}$ |
| Recall, | |
| Hit rate) | |
| True Negative Rate (Specificity, | $TNR = \frac{TN}{N} = \frac{TN}{TN + FP}$ |
| Selectivity) | |
| Positive Prediction Value (Precision) | $PPV = \frac{TP}{TP + FP}$ |
| Negative Predictive Value | $NPV = \frac{TN}{TN + FN}$ |
| False Possitive Rate | $FPR = \frac{FP}{N} = \frac{FP}{FP + TN}$ |
| False Negative Rate | $FNR = \frac{FN}{P} = \frac{FN}{FN + TP}$ |
| False Discovery Rate | $FDR = \frac{FP}{FP + TP} = 1 - PPV$ |
| False Omission Rate | $FOR = \frac{FN}{FN + TN} = 1 - NPV$ |
| Accuracy | $ACC = \frac{TP + TN}{P + N}$ |
| F1 Score | $F 1 = 2 \cdot \frac{PPV \cdot TPR}{PPV + TPR}$ |

B. Ablation Study

To effectively evaluate the performance of the proposed model, the results of VBGAN and vanilla DenseNet121 were compared in two setups: binary classification and multi-label classification. Table 2 shows the F1 score and other standard metrics on the test set of 100 positive COVID-19 images and 2,209 negative images. This test set is rather difficult due to its heavy imbalance property between the positive and negative samples. A model with decent performance on this test set would demonstrate a reliable True Negative Recall (Specificity) as it saves workload for False Positive cases. As shown in Table 2, the baseline DenseNet121 with 4-class prediction yields better results in the COVID-19 F1 score (0.7644 versus 0.7513) compared to the DenseNet121 binary mode. With the support from generative models in VBGAN, the F1 score metric improves to 0.7894 (for binary setup) and 0.8 (for the multi-label setup). Note that all models' scores are taken at a threshold of 0.5. Initially, the generator acts as a regularizer, generating CXR images with noisy labels. As training progresses, the generator's role becomes that of a data upsampler generating fake COVID image for the feasibility of classifier optimization. Adding fake images into classification training loop also helps increase the sensitivity (TPR) of the classifier to COVID images; compares to the baselines, and VBGAN allows the classifier to consistently achieve 4-5% increases in the number of the positive case it can recognize. In terms of specificity (TNR), VBGAN exceeds the performance of vanilla DenseNet121 by a small margin. Though comparable, VBGAN in a multi-label setup is slightly better than its binary setup. The task of generating X-ray images of desirable classified features is somewhat harder to achieve in a multi-label setup. The generated images containing multi-label features are likely not as correct as those produced in binary-label setup, introducing noise to the fake labels used for training. These outcomes verify the initial hypothesis that larger distributions (normal distribution for image part and uniform distribution for the label part) can help to suppress the noisy labels come from the doctors' decision making distributions.

TABLE 2

| | Method | | | | | | |
|---|---|---|---|---|---|---|---|
| | CovidAID [39] | Deep-COVID [40] | CoroNet [41] | Baselines | | VBGANs | |
| | Backbone | | | | | | |
| | DenseNet121 | ResNet50 | Xception | DenseNet121 | DenseNet121 | DenseNet121 | DenseNet121 |
| | Output | | | | | | |
| | Multi | Multi | Multi | Binary | Multi | Binary | Multi |
| Population | 2309 | 2309 | 2309 | 2309 | 2309 | 2309 | 2309 |
| Conditional Positive | 100 | 100 | 100 | 100 | 100 | 100 | 100 |
| Conditional Negative | 2209 | 2209 | 2209 | 2209 | 2209 | 2209 | 2209 |
| Predicted Positive | 133 | 134 | 95 | 89 | 91 | 90 | 95 |
| Predicted Negative | 2176 | 2175 | 2214 | 2220 | 2218 | 2219 | 2214 |
| TP | 92 | 87 | 77 | 71 | 73 | 75 | 78 |
| TN | 2168 | 2162 | 2191 | 2191 | 2191 | 2194 | 2192 |
| FP | 41 | 47 | 18 | 18 | 18 | 15 | 17 |
| FN | 8 | 13 | 23 | 29 | 27 | 25 | 22 |
| TPR | 0.92 | 0.87 | 0.77 | 0.71 | 0.73 | 0.75 | 0.78 |
| TNR | 0.981440 | 0.978723 | 0.991852 | 0.991852 | 0.991852 | 0.993210 | 0.992304 |
| PPV | 0.691729 | 0.649254 | 0.810526 | 0.797753 | 0.802198 | 0.833333 | 0.821053 |
| NPV | 0.996324 | 0.994023 | 0.989612 | 0.986937 | 0.987827 | 0.988734 | 0.990063 |
| FPR | 0.018560 | 0.021277 | 0.008148 | 0.008148 | 0.008148 | 0.006790 | 0.007696 |
| FDR | 0.308271 | 0.350746 | 0.189474 | 0.202247 | 0.197802 | 0.166667 | 0.178947 |
| FNR | 0.08 | 0.13 | 0.23 | 0.29 | 0.27 | 0.25 | 0.22 |
| ACC | 0.978779 | 0.974015 | 0.982243 | 0.979645 | 0.980511 | 0.982676 | 0.983110 |
| F1 score | 0.789700 | 0.743590 | 0.789744 | 0.751323 | 0.764398 | 0.789474 | 0.800000 |

C. Comparison with Other Concurrent Work

A comparison is roughly made with other concurrent works, which are: CovidAID, Deep-COVID and CoroNet. These models have been fine-tuned on the training set for 300 epochs and evaluated on the test set. Deep-COVID shipped with two backbones: ResNet18 and ResNet50 are both also pre-trained from ImageNet and finetuned on the training set. It is empirically observed that the result from Deep-COVID (ResNet50) outperforms ResNet18. Intuitively, yet because ResNet50 is deeper than ResNet18 and hence results in better classification performance. CoroNet also presents an exciting approach that uses pre-trained Xception as its backbone on ImageNet. As is also shown in Table 2, CovidAID achieved the best Recall (0.92) compared to other and the apparatus of invention (0.78). This can be explained using the reason that CovidAID makes use of the large ImageNet and CheXpert in its pre-trained weight while the apparatus of invention leverage the checkpoint of DenseNet121 on ImageNet only. However, in terms of precision, the VBGAN models (both binary and multi setups) outperform the baselines and the other concurrent works. It is understandable because VBGANs avoid false positive predictions by using the generated positive samples. Consequently, in terms of the F1 score, a harmonic mean of the precision and recall, the VBGAN models obtain the highest value (0.8) compared to the others and the baselines models.

The present invention addresses the classification task as multi-label while ACGAN originally works on multi-class problems. The present invention generates high-resolution (up to 256×256) and high-quality CXR images, as opposed to moderate-resolution natural images in ACGAN.

Various advantages and effects of the present invention are not limited to those described above and may be easily understood in the detailed description of embodiments of the present invention.

The term “unit” used in the exemplary embodiment of the present invention means software or a hardware component, such as a field-programmable gate array (FPGA) or application-specific integrated circuit (ASIC), and a “unit” performs a specific role. However, a “unit” is not limited to software or hardware. A “unit” may be configured to be present in an addressable storage medium and may also be configured to run one or more processors. Therefore, as an example, a “unit” includes elements, such as software elements, object-oriented software elements, class elements, and task elements, processes, functions, attributes, procedures, subroutines, segments of program code, drivers, firmware, microcode, circuits, data, a database, data structures, tables, arrays, and variables. Elements and functions provided in “units” may be formed by coupling a smaller number of elements and “units” or may be subdivided into a greater number of elements and “units.” In addition, elements and “units” may be implemented to run one or more central processing units (CPUs) in a device or a secure multimedia card.

Although the embodiments have been mainly described above, they are only examples and do not limit the present invention. Those of ordinary skill in the art may appreciate that a variety of modifications and applications not presented above can be made without departing from the essential characteristic of the embodiments. For example, each element specifically represented in the embodiments may vary. Also, it should be construed that differences related to such modifications and applications fall within the scope of the present invention defined in the following claims.

What is claimed is:

1. An apparatus for classifying a medical image, the apparatus comprising:
a database configured to store a first image;
a generator configured to generate a second image on the basis of a latent vector which is a concatenation of noise information having a certain size and random uniform class labels of a plurality of diseases;
a discriminator configured to receive the first image and the second image and attempt to recognize the first image and the second image as a real image and a fake image; and
a classifier configured to classify the first image and the second image.

2. The apparatus of claim 1, wherein the noise information has a size of 16 dimensions and is generated on the basis of a normal distribution.

3. The apparatus of claim 1, wherein the random uniform class labels of the plurality of diseases are random uniform class labels of coronavirus disease 2019 (COVID-19), airspace opacity, consolidation, and pneumonia and have a value of 0 for negative cases of the diseases and a value of 1 for positive cases of the diseases.

4. The apparatus of claim 1, wherein the discriminator calculates a probability distribution with relation to the second image.

5. The apparatus of claim 1, wherein the generator and the discriminator are implemented as progressive growing generative adversarial networks (GANs).

6. The apparatus of claim 1, wherein the classifier is implemented as DenseNet121.

7. The apparatus of claim 6, wherein in the classifier, the number of output neurons is set differently depending on a classification type.

8. The apparatus of claim 7, wherein the classifier sets the number of output neurons to 1 when the classification type is a binary label classification and set the number of output neurons to 4 when the classification type is a multi-label classification.

9. The apparatus of claim 8, wherein all of the activations of the classifier and the discriminator are replaced by Leaky ReLU.

10. The apparatus of claim 9, wherein the classifier sets a leaky coefficient to 0.02.

11. The apparatus of claim 10, wherein a final layer of the classifier uses a logistic sigmoid function.

12. The apparatus of claim 1, wherein the generator, the discriminator, and the classifier are trained on the basis of the following formula:
$${min}_{{θ}_{G} , {θ}_{C}} {max}_{{θ}_{D}} L ( C ) + λ ( V ( G , D ) + L ( G , C ) ) .$$
where L(C) denotes a classification loss, V(G, D) denotes an adversarial loss, L(G, C) denotes a classification-driven generative loss, and λ denotes a hyperparameter.

13. The apparatus of claim 12, wherein the hyperparameter is 0.1.

14. The apparatus of claim 12, wherein the hyperparameter is 1 when optimizing discriminator and generator.

15. The apparatus of claim 1, further comprising a diagnostic unit configured to make a disease diagnosis from an image of a patient on the basis of the classified first image and second image.
